(19) United States
(12) Patent Application Publication
(10) Pub. No.: US 2006/0233711 A1
(43) Pub. Date: Oct. 19, 2006

(54) Method for treating conditions associated with fear memory

(76) Inventor: Min Zhuo, Toronto (CA)

Correspondence Address: GOWLING, LAFLEUR HENDERSON LLP, ONE MAIN STREET WEST, HAMILTON, ON, L8P 4Z5, CA

(21) Appl. No.: 11/399,476

(22) Filed: Apr. 7, 2006

Related U.S. Application Data
(60) Provisional application No. 60/669,544, filed on Apr. 8, 2005.

(51) Int. Cl. A61K 49/00; G01N 33/53

(52) U.S. Cl. 424/9.2; 435/7.1

(57) ABSTRACT

A method of treating conditions associated with fear memory in a mammal is provided which includes selective inhibition of a GluR6 receptor. Methods of screening for compounds useful to treat such conditions are also provided.

### FIELD OF THE INVENTION

The present invention relates to a method of treating conditions associated with fear memory and in particular to treatment methods which comprise inhibition of the GluR6 receptor.

### BACKGROUND OF THE INVENTION

Kainate receptors (KARs) are composed of five different subunits (Hollmann and Heinemann, 1994). These include glutamate receptor 5 (GluR5), GluR6, and GluR7 subunits, which form functional homomeric receptors, and KA1 and KA2, which combine in heteromeric receptors but do not form functional ion channels on their own. In the periphery and spinal cord, KARs play an important role in sensory transmission. KARs are located on sensory afferent fibers and dorsal root ganglion (DRG) cells (Partin et al., 1993; Tolle et al., 1993; Procter et al., 1998; Hwang et al., 2001; Kerchner et al., 2001a; Kerchner et al., 2002). In the spinal cord, they are located on the postsynaptic membrane of dorsal horn neurons and contribute to synaptic responses to high-threshold primary afferent fiber stimulation (Li et al., 1999). KARs are also present presynaptically on the primary afferent fibers themselves (Davies et al., 1979; Huettner, 1990), where they can regulate glutamate release in the spinal cord (Kerchner et al., 2001b). Furthermore, presynaptic KARs biphasically regulate inhibitory transmission in the spinal dorsal horn (Kerchner and Zhuo, 2002). The deletion of GluR5 abolished KAR function in DRG neurons (Kerchner et al., 2002). However, glutamate-mediated sensory synaptic transmission is normal in spinal cord slices of GluR5 and GluR6 knockout mice (Youn and Randic, 2004). Behavioral responses to both formalin and Complete Freund's adjuvant (CFA) are reduced when animals are treated with the selective GluR5 receptor antagonist LY382884 (Simmons et al., 1998; Guo et al., 2002), indicating a role for GluR5 in pain transmission. These findings suggest that GluR5 is essential for KAR-mediated responses in DRG cells and for presynaptic regulation in the spinal dorsal horn.

KARs are also distributed in higher brain structures, such as the amygdala and related cortical areas (Hollmann and Heinemann, 1994; Li et al., 2001). In the hippocampus, KARs contribute to presynaptic regulation and postsynaptic responses to repetitive stimulation (Frerking and Nicoll, 2000; Kullmann, 2001; Huettner et al., 2002; Lerma, 2003). GluR5-containing KARs in the amygdala contribute to heterosynaptic facilitation induced by prolonged low-frequency stimulation (Li et al., 2001). The role of the KARS in fear memory has yet to be elucidated.

KARs are believed to be important for learning and memory, in part due to their roles in synaptic plasticity in the hippocampus and amygdala (Frerking and Nicoll, 2000; Kullmann, 2001; Huettner et al., 2002; Lerma, 2003). Early contextual and auditory fear memory is mediated by the hippocampus and/or amygdala, while late contextual memory may be mediated by cortical areas (Sutherland and McDonald, 1990). Despite in vitro electrophysiological evidence of KARs in the amygdala, little information is available about the role(s) of KARs in learning and memory.

It would be desirable to identify the role of each KAR in learning and memory in order that treatments for medical conditions associated with defects in these areas could be developed.

### SUMMARY OF THE INVENTION

In one aspect of the present invention, there is provided a method of treating a condition associated with fear memory in a mammal, wherein the method comprises the step of selectively inhibiting GluR6 receptors in the mammal.

In another aspect of the invention, there is provided a novel use for a GluR6 inhibitor in the treatment of a condition associated with fear memory in a mammal.

A composition for use in treating conditions associated with fear memory in a mammal is also provided comprising a GluR6 inhibitor combined with a pharmaceutically acceptable carrier.

An article of manufacture is also provided comprising packaging containing a composition for use in treating a condition associated with fear memory, said packaging comprising a label indicating that the composition is for use in treating a condition associated with fear memory, said composition comprising a GluR6-inhibiting compound.

In another aspect of the invention, there is provided a method of screening for a drug candidate useful to treat in a mammal a condition associated with fear memory, comprising:
assaying a compound for GluR6 interaction;
assaying the compound for interaction with at least one other kainate receptor;
comparing the interaction of the compound with GluR6 and with the other kainate receptor, wherein interaction with GluR6 but not with the other kainate receptor indicates that the compound is a drug candidate.

In another aspect, there is provided a method of screening for a drug candidate useful to treat in a mammal a condition associated with fear memory. The method comprises:
1) incubating a first mixture of labelled ligand with a GluR6 receptor-producing cell or with a membrane preparation derived therefrom followed by incubation with a drug candidate;
2) incubating a second mixture of labelled ligand with another kainate receptor-producing cell or with a membrane preparation derived therefrom followed by incubation with a drug candidate; and
3) measuring the amount of labelled ligand in the first and second mixtures that is displaced following incubation with the drug candidate, wherein a concentration of displaced labelled ligand in the first mixture as compared with the second mixture is indicative of selective GluR6 inhibition.

In another aspect, a method of screening for a drug candidate useful to treat in a mammal a condition associated with fear memory is provided. The method comprises:
1) incubating a mixture of ligand with a GluR6 receptor-producing cell or with a membrane preparation derived therefrom to obtain a ligand-induced electrical current across said cell or membrane followed by incubation with a drug candidate;
2) incubating a mixture of ligand with another kainate receptor-producing cell or with a membrane preparation derived therefrom to obtain a ligand-induced electrical current across said cell or membrane followed by incubation with a drug candidate; and
3) comparing the effect of the drug candidate on GluR6 and the other kainate receptor, wherein a decrease in electrical current across the GluR6-encoding cell or membrane while little or no decrease occurs in the electrical current across the other kainite receptor-producing cell or membrane is indicative of selective GluR6 inhibition.

These and other aspects of the invention will become apparent by reference to the following detailed description and figures.

### BRIEF DESCRIPTION OF THE DRAWINGS

FIG. 1 illustrates the results of contextual fear conditioning in GluR6 or GluR5 knockout and wild-type mice at 1 hr, 1, 3, 7 and 14 days after training (A/B), as well as the results of auditory fear conditioning in GluR6 or GluR5 knockout and wild-type mice at 1 hr, 1, 3, 7 and 14 days after training (C/D);

FIG. 2A illustrates the placement of stimulating and recording electrodes in the amygdala;

FIG. 2B illustrates the TBS (indicated by the arrow) induced synaptic potentiation in the amygdala of wild-type (n=11 slices/9 mice) but not GluR6 knockout mice (n=8 slices/6 mice). Inset: representative records of the fEPSP before (Pre) and 40 min after (Post) TBS;

FIG. 2C illustrates the TBS (indicated by the arrow) induced synaptic potentiation in the amygdala of GluR5 knockout mice (n=6 slices/6 mice). Inset: representative records of the fEPSP before (Pre) and 40 min after (Post) TBS;

FIG. 3A shows that injection of depolarizing current into a neuron induced action potentials that showed significant firing frequency adaptation;

FIG. 3B shows that LTP was induced in the LA of wild type littermates (n=9 slices/5 mice), but not in GluR6 knockout mice (n=11 slices/6 mice). Inset: representative records of EPSCs recorded during baseline collection and 20 minutes after the pairing training (arrow);

FIG. 3C shows that paired training induced LTP in GluR5 knockout mice (n=9 slices/7 mice). Inset: representative records of EPSCs recorded during baseline collection and 20 minutes after the pairing training (arrow);

FIG. 4A shows the EPSCs recorded in the presence of AP5, 5 min after addition of SYM2206 (100 μM) and 2 min after addition of CNQX (20 μM) in a LA neuron;

FIG. 4B graphically illustrates the peak EPSC amplitude versus time for the traces shown in FIG. 4A;

FIG. 4C shows the SYM2206-insensitive EPSC scaled to the peak of the SYM2206 sensitive EPSC;

FIG. 4D graphically illustrates the time constant of EPSC decay versus the rising time (10-90%) for EPSCs mediated by SYM2206 sensitive (square) and SYM2206 insensitive (circle) component;

FIG. 5A is a diagram showing the placement of stimulating and recording electrodes in the auditory cortex;

FIG. 5B graphically illustrates that TBS failed to induce significant potentiation in GluR6 knockout mice (n=5 slices/5 mice) compared to wild-type mice (n=7 slices/7 mice). Inset: representative records of the fEPSP before (Pre) and 40 min after (Post) TBS; and

FIG. 5C graphically illustrates that TBS induced significant potentiation in GluR5 knockout mice (n=7 slices/5 mice). Inset: representative records of the fEPSP before (Pre) and 40 min after (Post) TBS.

### DETAILED DESCRIPTION OF THE INVENTION

A novel method for treating conditions associated with fear memory in mammals is provided. The method comprises the selective inhibition of GluR6 in a mammal in need of treatment.

The invention results from the determination that the selective inhibition of GluR6 results in significant reduction of contextual and auditory fear memory following classic fear memory tests which include measure of response to noxious stimuli following fear conditioning. In contextual fear conditioning, a subject forms an association between a distinctive context and an aversive event that takes place in that context. When placed back into the context, the subject exhibits a range of conditioned fear responses, including freezing. Auditory fear conditioning is similar to contextual fear conditioning with the exception that the distinctive context is replaced by a distinctive auditory event with which an association to an aversive event is formed.

The terminology “condition associated with fear memory” is used herein to encompass medical conditions that may develop in a mammal that are related to the emotion of fear including mild, moderate and severe psychoses and/or phobias that have an impact on normal day-to-day activities and social interactions. Fear is an emotion caused by the threat of danger, pain or harm, and when linked to the memory of an aversive event(s), may be exhibited in the form of anxiety and/or depression.

The term “treatment” refers to the prophylaxis, amelioration or elimination of at least one condition associated with fear memory as defined above.

The term “mammal” is used herein to denote any mammal, including but not limited to, humans.

The treatment of a condition associated with fear memory in accordance with the invention comprises selective inhibition of GluR6 receptors in a mammal suffering from such a condition. In one embodiment of the invention, this treatment entails the administration to the mammal of a therapeutically effective amount of a selective GluR6 inhibitor. A GluR6 inhibitor is a substance capable of at least reducing the functional activity of GluR6 receptors in a mammal to an extent that is useful to reduce or prevent fear memory. The term “selective” as it is used with respect to a GluR6 inhibitor refers to the fact that the inhibitor acts primarily on GluR6 receptors having little or no effect on surrounding or similar receptors, including for example, other KAR receptors such as GluR5, GluR7, KA1 and KA2 receptors.

The GluR6 inhibitor may be administered in a treatment protocol as set out above, alone, or alternatively may be administered in combination with a pharmaceutical carrier to form a composition. The term “pharmaceutically acceptable” refers to the acceptability of the carrier for administration to a mammal in the dosage form contemplated for use in the treatment protocol which does not affect the therapeutic activity of the active GluR6 inhibitor. Suitable such carriers include, but are not limited to, sugars, starches, cellulose and derivatives thereof, wetting agents, lubricants such as sodium lauryl sulfate, stabilizers, tabletting agents, anti-oxidants, preservatives, coloring agents and flavouring agents. Reference may be made to “Remington's Pharmaceutical Sciences”, 17th Ed., Mack Publishing Company, Easton, Pa., 1985, for other carriers that would be suitable for combination with a GluR6 inhibitor to render a composition in accordance with the invention.

As will be appreciated, the pharmaceutical carriers used to prepare compositions in accordance with the present invention will depend on the administrable dosage form to be used. Generally, dosage forms suitable for oral administration, including, for example, tablets, capsules, powders and fluid compositions, such as solutions or suspensions, are contemplated; however, inhalable aerosols, injectables, creams for external application and formulations for application via suppository may also be suitable.

In the treatment method of the present invention, a therapeutically effective amount of a selective GluR6 inhibitor is administered to a mammal in need of treatment. The term “therapeutically effective” refers to a GluR6 inhibitor that is effective to inhibit GluR6 receptors in the mammal being treated while not otherwise having significant adverse side effects to the mammal. While side effects to a certain degree can be tolerated and may even be acceptable, side effects which are in any way debilitating, disease-causing or life-threatening are clearly unacceptable. Dosages of a GluR6 inhibitor that are suitable for use in the treatment of a condition associated with fear memory can readily be determined in well-established, appropriately controlled trials as would be appreciated by one of skill in the art. It is anticipated that a dosage of GluR6 inhibitor in the range of about 1-10 mg/kg per day may be useful to treat a condition associated with fear memory as defined above.

In another aspect, an article of manufacture comprising packaging containing a composition for use in treating a condition associated with fear memory is provided. The packaging is labelled to indicate that the composition is for use in treating a condition associated with fear memory. The composition comprises a selective GluR6 inhibitor combined with at least one pharmaceutically acceptable carrier as detailed above

The packaging may be any material suitable to package a pharmaceutical composition including, for example, a bottle or other suitable container made of material that protects the contents thereof from degradation, such as moisture, sunlight, contamination, etc.

While some GluR6 inhibitors or antagonists have been identified, candidate drug compounds which selectively interact with or inhibit GluR6, and thus may be suitable for use to treat conditions associated with fear memory in mammals, may be elucidated using the screening methods provided in another aspect of the present invention.

Mammalian cells encoding GluR6 in functional form may be used to determine whether a drug candidate selectively interacts with or inhibits GluR6, provided that the selected cell line does not encode other KARs as this would not yield useful results. The construction of such cell lines is achieved using methods well-established in the field which comprise introduction into a selected host cell of a recombinant DNA expression vector in which DNA coding for a secretable form of GluR6 , i.e. a form bearing either its native signal peptide or a functional, heterologous equivalent thereof, is associated with expression controlling elements that are functional in the selected host to drive expression of the receptor-encoding DNA, and thus elaborate the desired receptor protein. Such cells are herein characterized as having the receptor-encoding DNA incorporated “expressibly” therein. The receptor-encoding DNA is referred to as “heterologous” with respect to the particular cellular host if the DNA is not naturally found in the particular host. Methodology for constructing such KAR-encoding cell lines in detailed in, for example, U.S. Pat. No. 6,013,768. Such cell lines are also available at the ATCC (American Tissue Culture Collection) in Bethesda, U.S.A.

For incorporation into the recombinant DNA expression vector, DNA coding for the desired GluR6 receptor, e.g. the GluR6 receptor or a kainate-binding variant thereof, can be obtained by applying selected techniques of gene isolation or gene synthesis. Kainite-binding receptors such as GluR6 are encoded within the genome of human brain tissue, and can therefore be obtained by careful application of conventional gene isolation and cloning techniques. Automated techniques of gene synthesis and/or amplification can also be performed to generate DNA encoding GluR6. Application of automated synthesis may require staged gene construction, in which regions of the gene up to about 300 nucleotides in length are synthesized individually and then ligated in correct succession for final assembly due to the size of GluR6 DNA. In order to conduct such automated techniques, reference may be made to Nature. 1991 Jun. 27; 351(6329): 745-8 which discloses the sequence of human GluR6 DNA. The application of automated gene synthesis techniques provides an opportunity to generate sequence variants of naturally occurring GluR6 that have corresponding functional characteristics, but which may provide a desirable structural property, for example, increased stability. GluR6 variants may include, for example, one or more single amino acid substitutions, deletions or additions. Since it will for the most part be desirable to retain in any variant native GluR6 activity and natural ligand binding profile, it is desirable to limit amino acid substitutions to the so-called conservative replacements in which amino acids of like charge are substituted, and to limit substitutions to those sites less critical for receptor activity. Use of such variants in screening assays according to the present invention may be appropriate provided that the GluR6 variant retains native GluR6 activity and binding profile so as to provide accurate data with respect to candidate drug compounds.

For use in screening assays, GluR6-encoding cells or GluR6-encoding membrane preparations derived from such cells may be used. The membrane preparations typically provide a more convenient substrate for ligand binding experiments, and are therefore preferred as binding substrates. To prepare membrane preparations for use in screening assays, frozen intact GluR6-expressing cells are homogenized while in cold water suspension and centrifuged. The collected pellet is then washed in cold water, and dialyzed to remove endogenous ligands, such as glutamate, that would otherwise compete for binding in the assays. The dialyzed membranes may be used in screening assays, or stored in lyophilized form for future use. Alternatively, intact, fresh cells harvested about two days after transient transfection or after about the same period following fresh plating of stably transfected cells, can be used in screening assays. When fresh cells are used, the cells must be harvested by more gentle centrifugation so as not to damage them, and all washing must be done in a buffered medium, for example in phosphate-buffered saline, to avoid osmotic shock and rupture of the cells.

The binding of a candidate compound to a GluR6 receptor of the invention is evaluated typically using a predetermined amount of cell-derived membrane (measured for example by protein determination), generally from about 25 ug to 100 ug. Competitive binding assays are generally useful to evaluate the affinity of a test compound relative to an endogenous ligand, such as kainate. This competitive binding assay can be performed by incubating the membrane preparation with radiolabelled kainate, for example [3H]-kainate, in the presence of unlabelled test compound added at varying concentrations. Following incubation, either displaced or bound radiolabelled kainate can be recovered and measured, to determine the relative binding affinities of the test compound and kainate for the particular receptor used as substrate. In this way, the affinities of various compounds for the kainate-type receptors can be measured.

In one embodiment, a method of screening for a GluR6 selective candidate compound useful to treat in a mammal a condition associated with fear memory is provided comprising assaying a compound for interaction (including binding or other inhibitory activity) with GluR6, and comparing that to the binding/inhibitory activity of the compound against at least one other kainate receptor, such as GluR5 or GluR7. Inhibition of GluR6 but not the other selected kainate receptor(s) indicates that the compound is a GluR6-selective drug candidate.

Such a method of screening may comprise, for example, incubating a first mixture of labelled ligand with a GluR6 receptor-producing cell or with a membrane preparation derived therefrom followed by incubation with a drug candidate. A second mixture of labelled ligand is independently incubated with another kainate receptor-producing cell or with a membrane preparation derived therefrom followed by incubation with a drug candidate. As this is a competitive binding assay, the amount of displaced labelled ligand in the first and second mixtures following incubation with the drug candidate can be measured to determine the effect of the drug candidate on ligand binding. An increased concentration of displaced labelled ligand in the first mixture as compared with the second mixture is indicative of selective GluR6 inhibition.

GluR6 receptors are functional in an electrophysiological context. Therefore, candidate drug compounds can be screened for their ability to modulate ion channel activity. Therefore, the present invention also provides a screening technique which detects the effect of a candidate drug compound on a GluR6 receptor in the presence of a GluR6 ligand such as kainate. The technique includes the steps of incubating a mixture of ligand with a GluR6 receptor-producing cell or with a membrane preparation derived therefrom to obtain a ligand-induced electrical current across said cell or membrane, incubating the ligand mixture with a candidate compound, and determining whether there is inhibition of the GluR6 receptor in the presence of the candidate compound, wherein a decrease in electrical current across the cell or membrane is indicative of inhibition.

Screening assays may also be performed using cells, for example, Xenopus oocytes, that yield functional membrane-bound receptor following introduction of receptor-encoding messenger RNA in well-established techniques. Following the injection of nL volumes of an RNA solution, the oocytes are left to incubate for up to several days, and are then tested in either intact form or as a membrane preparation for the ability to bind a particular ligand molecule supplied in a bathing solution. Since functional KA receptors act in part by operating a membrane channel through which ions may selectively pass, the impact of a candidate drug compound on a functioning GluR6 receptor can be measured in terms of inhibition of electrical current which is monitored by microelectrodes inserted into the cell or placed on either side of a cell-derived membrane preparation using the “patch-clamp” technique.

In another embodiment, an electrophysiological method of screening for a GluR6 selective drug candidate is provided based on the selectivity of the drug candidate to modulate ion channel activity in GluR6. The method comprises incubating a mixture of kainite receptor ligand with a GluR6 receptor-producing cell or with a membrane preparation derived therefrom to obtain a ligand-induced electrical current across said cell or membrane. This incubation is followed by incubation with a drug candidate. For comparison, a mixture of the ligand is incubated with another kainate receptor-producing cell or with a membrane preparation derived therefrom to obtain a ligand-induced electrical current across said cell or membrane followed by incubation with the drug candidate. The effect of the drug candidate on GluR6 and the other kainate receptor is compared. A decrease in electrical current across the GluR6-encoding cell or membrane while little or no decrease in the electrical current across the other kainate receptor-producing cell or membrane is indicative of selective GluR6 inhibition.

Suitable animal models may also be used to screen for selective GluR6 inhibitors or antagonists, including wild-type and genetically modified animal models. In one embodiment, for example, wild-type mice, appropriately conditioned for experimentation, may be used. The mice are exposed to fear conditioning, such as contextual and/or auditory fear conditioning, as outlined in the specific examples. Following fear conditioning, an appropriate dosage of a drug candidate compound is administered to a treatment group, while a control group is given a placebo. The treatment and control groups, following an appropriate incubation period, are then exposed to a setting suitable to trigger a fear-related response in accordance with the fear conditioning. Lack of response in the treatment group in comparison to the control group is indicative that the candidate drug compound is a selective GluR6 inhibitor. GluR6-knockout mice, as described in the examples that follow, may also be useful to confirm GluR6 selectivity of a candidate compound. A compound determined to inhibit GluR6 in vitro may be screened using such knockout mice. Lack of effect on a GluR6 knockout confirms that the activity of the compound is restricted to GluR6, and thus, GluR6-selective.

It will be understood by those of skill in the art that embodiments may exist that are not described herein, but which fall within the scope of the appended claims.

All references are incorporated herein by reference.

Embodiments of the present invention are described by reference to the following specific examples which are not to be construed as limiting.

### EXAMPLES

### Methods and Materials

Adult male mice (8-12 weeks old) were used for all experiments. GluR5 and GluR6 knockout mice were gifts from Dr. Stephen F. Heinemann (Salk Institute, San Diego, Calif., USA) (Mulle et al., 1998; Mulle et al., 2000). Mice were housed on a 12 hour light: dark cycle with ad libitum access to food and water. GluR5 and GluR6 knockout mice were maintained on a mixed 129Sv×C57BL/6 background and 129sv/C57BL/6 mice from Taconic were used as controls. Additional experiments were performed on GluR5 and GluR6 wild-type littermates and no significant difference was found when compared to 129sv/C57BL/6 mice from Taconic. The Animal Care and Use Committee at the University of Toronto approved all mouse protocols. Because GluR5 and GluR6 knockout mice are visually indistinguishable, all experiments were performed blind to the genotype.

Fear conditioning was performed in an isolated shock chamber (Med Associates, St. Albans, Vt., USA). An experimenter blind to the genotype manually scored freezing responses (total immobility aside from respiration) every 10 sec. The conditioned stimulus (CS) was an 85 dB sound at 2,800 Hz, and the unconditioned stimulus (US) was a continuous scrambled foot shock at 0.75 mA. After 2 min of habituation, animals received the CS/US pairing (a 30 sec tone (CS) and a 2 sec shock (US) starting at 28 sec, three shock-tone pairings were delivered at 30 sec intervals) and the mice remained in the chamber for an additional 30 sec to measure immediate freezing. One hour, 1, 3, 7 and 14 days after training, each mouse was placed back into the shock chamber and the freezing response was recorded for 3 min (contextual conditioning). Subsequently, the mice were placed into a novel chamber and monitored for 3 min before the onset of a tone identical to the CS, which was delivered for 3 min, and freezing responses were recorded (auditory conditioning).

Slice Electrophysiology

Animals were anesthetized with halothane. Transverse slices (400 □□) of the amygdala and auditory cortex were rapidly prepared using a vibratome (Vibratome Series 1000, Technical Products International INC, St. Louis, Mo., USA) and maintained in an interface chamber at 30° C., where they were subfused with artificial cerebrospinal fluid (ACSF) consisting of (in mM): 124 NaCl, 4.4 KCl, 2.0 CaCl₂, 1.0 MgSO₄, 25 NaHCO₃, 1.0 NaH₂PO₄, and 10 glucose, bubbled with 95% O₂ and 5% CO₂. Slices were kept in the recording chamber for at least two hours prior to the start of experiments (Wei et al., 2002). In amygdala slices, a bipolar tungsten stimulating electrode (Micro Probe, Inc., Md., USA) was placed in the ventral striatum, and an extracellular recording electrode (3-12 MΩ filled with ACSF) was placed in the lateral amygdala. In auditory cortical slices, a bipolar tungsten stimulating electrode was placed in layer V, and extracellular field potentials were recorded using a glass microelectrode placed in layer II/III. Synaptic responses were elicited every 50 sec by electrical stimulation (200 μsec duration). After obtaining stable recordings for at least 15-20 min, five trains of theta burst stimulation (TBS), which consisted of five bursts (four pulses at 100 Hz) of stimuli delivered every 200 ms at the same intensity, were applied.

Whole-Cell Patch Clamp Recordings

Transverse slices (300 □M) of the amygdala were transferred to a room temperature submerged recovery chamber with oxygenated (95% O₂ and 5% CO₂) ACSF solution as described above. After a one hour recovery, slices were placed in a recording chamber on the stage of an Axioskop 2FS microscope (Zeiss) equipped with infrared DIC optics for visualized whole-cell patch clamp recordings. Excitatory postsynaptic currents were recorded from cells in the lateral amygdala with an Axon 200B amplifier (Axon Instruments, Calif., USA). Electrical stimulations (200 μsec duration) were delivered by a bipolar tungsten stimulating electrode placed in the internal capsule (thalamic inputs) (Tsvetkov et al., 2004). EPSCs were induced by repetitive stimulations at 0.02-0.05 Hz and neurons were voltage clamped at −70 mV. In all experiments, the stimulus intensity was adjusted to produce synaptic responses with an amplitude of 70-100 pA. In LTP experiments, the recording pipettes (3-5 MΩ were filled with solution containing (mM): 145 K-gluconate, 5.0 NaCl, 1.0 MgCl₂, 0.2 EGTA, 10 HEPES, 2.0 Mg-ATP, and 0.1 Na₃-GTP (adjusted to pH 7.2 with KOH; 280-300 mOsmol). Picrotoxin (100 □M) was always present in the perfusion solution (ACSF) to block GABA_{A} receptor-mediated inhibitory synaptic currents. After obtaining a stable EPSC for at least 10 min, LTP was induced by 80 pulses at 2 Hz paired with postsynaptic depolarization at +30 mV (Tsvetkov et al., 2004). AMPA receptor-mediated components of EPSCs were pharmacologically isolated in ACSF containing: AP-5 (50 □M) and picrotoxin (100 □M). To detect KAR-mediated EPSCs, SYM2206 (100 □M) and CNQX (20 □M) were sequentially applied through bath solution. The patch electrodes contained (in mM) 120 cesium gluconate, 5.0 NaCl, 1.0 MgCl₂, 0.5 EGTA, 2.0 MgATP, 0.1 Na₃GTP, 10 HEPES, 2.0 QX-314 (adjusted to pH 7.2 with CsOH; 280-300 mOsmol). Access resistance was 15-30 M [symbol] and monitored throughout the experiment. Data were discarded if access resistance changed more than 15% during an experiment.

Drugs

All chemicals and drugs were obtained from Sigma (St. Louis, Mo., USA), except for (±)-4-(4-aminophenyl)-1,2-dihydro-1-methyl-2-propylcarbamoyl-6,7-methylenedioxyphthalazine (SYM2206) and lidocaine N-methyl bromide quaternary salt (QX-314) which were from Tocris Cookson (Ellisville, Mo., USA).

### Results

Results were expressed as mean ±SEM. Statistical comparisons were performed using one- or two-way analysis of variance (ANOVA) using the Student-Newmann-Keuls test for post-hoc comparisons. In all cases, p<0.05 was considered statistically significant.

Fear Memory After Classic Conditioning

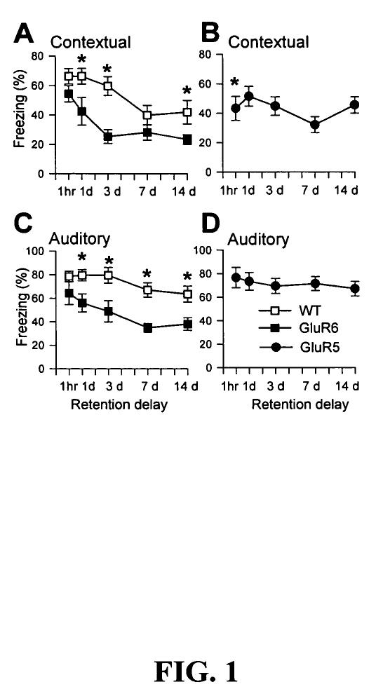

To determine if deletion of either GluR5 or GluR6 affected long-term fear memory, fear conditioning was performed in wild-type and knockout mice (Davis et al., 1997; LeDoux, 2000; Maren, 2001). Contextual and auditory fear memory was measured at 1 hr, 1, 3, 7 and 14 days after conditioning (Wei et al., 2002). There was no significant difference in freezing responses immediately after training among wild-type (n=8 mice, 39.6±4.1%), GluR5 (n=7, 33.3±6.3%) and GluR6 (n=8, 31.0±8.5%) knockout mice, suggesting that the deletion of GluR5 and GluR6 did not cause any developmental defect that would interfere with the shock-induced freezing response. This reinforces our assertion that acute nociceptive thresholds in GluR5 and GluR6 knockout mice were unaffected by the genetic manipulation. GluR6 knockout mice showed a significant reduction in both contextual and auditory fear memory at early (1 and 3 days), as well as later (1 and 2 weeks) time points after conditioning (FIG. 1). In contrast, contextual and auditory fear memory was unaltered in GluR5 knockout mice, except for a small reduction at one early time point (1 hour after conditioning) (FIG. 1B).

KAR Mediated Synaptic Plasticity in the Lateral Amygdala

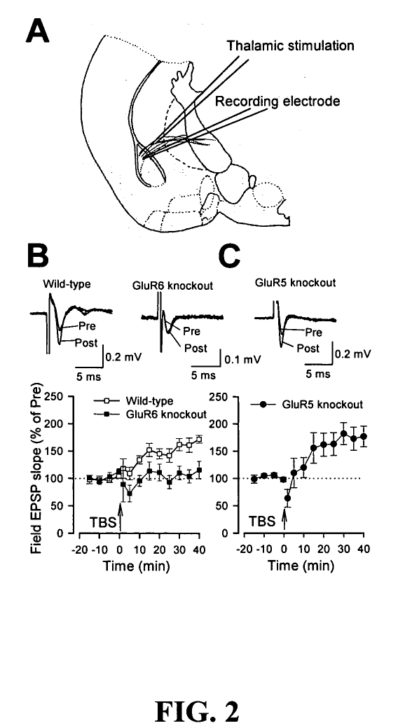

Synaptic plasticity, including long-term potentiation (LTP), is thought to be important for fear learning and memory (Bliss and Collingridge, 1993; McKernan and Shinnick-Gallagher, 1997; Rogan et al., 1997; Maren, 1999; Tsvetkov et al., 2002). Due to the significant reduction of fear memory in GluR6 knockout mice, synaptic potentiation in the amygdala, a structure known to be important in fear memory, was examined. Synaptic potentiation at ‘thalamic’ input synapses to the lateral amygdala (LA) was examined by placing a stimulating electrode in the ventral striatum (see FIG. 2A) (Wei et al., 2002). For these experiments, five trains of theta burst stimulation (TBS) (Frankland et al., 2001; Wei et al., 2002) were used. In wild-type mice, TBS induced significant synaptic potentiation (164.9±7.9%; n=11 slices/9 mice; p<0.05 compared to baseline; FIG. 2B). However, synaptic potentiation in slices of GluR6 knockout mice was significantly reduced or blocked (103.4±17.2%; n=8 slices/6 mice; p<0.01 compared to wild-type mice). In slices of GluR5 knockout mice (173.9±19.7%; n=6 slices/6 mice; p=0.33), TBS induced significant synaptic potentiation similar to that of wild-type mice (FIG. 2C).

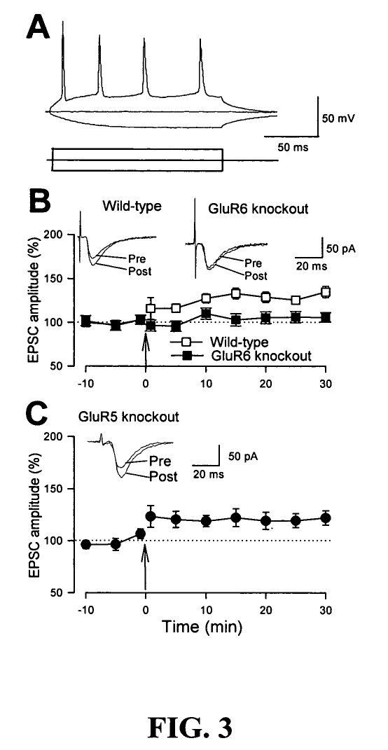

Whole-cell patch-clamp recordings from visually identified pyramidal neurons in the LA were also performed. Depolarizing currents were injected into the neuron which induced repetitive action potentials with a frequency adaptation that is typical of the firing pattern of pyramidal neurons (FIG. 3A) (Tsvetkov et al., 2002). Excitatory postsynaptic currents (EPSCs) were recorded in response to stimulation of the thalamic input. LTP was induced by pairing presynaptic stimulation with postsynaptic depolarization (see Methods). LTP was induced with paired pulses within 15 minutes after establishing the whole-cell configuration, since it was not possible to induce LTP of whole-cell EPSCs in amygdala synapses after 20 minutes (Tsvetkov et al., 2002). In wild-type littermate mice, the paired training induced long-lasting potentiation of responses (130.4%±6.1%; n=9 slices/5 mice; p<0.05 compared to baseline; FIG. 3B). However, synaptic potentiation in slices of GluR6 knockout mice was completely blocked (105.4%±6.5%; n=11 slices/6 mice; p<0.05 compared to wild-type; FIG. 3B). In slices of GluR5 knockout mice, the paired training still produced synaptic potentiation (120.0%±3.6%; n=9 slices/7 mice; p=0.17 compared to wild-type littermates, FIG. 3C). The fact that synaptic potentiation was selectively decreased in GluR6 knockout mice supports results from the fear memory study and suggests that the GluR6 subunit may play an important role in contextual and auditory fear memory formation.

KAR Mediated EPSCs in the LA

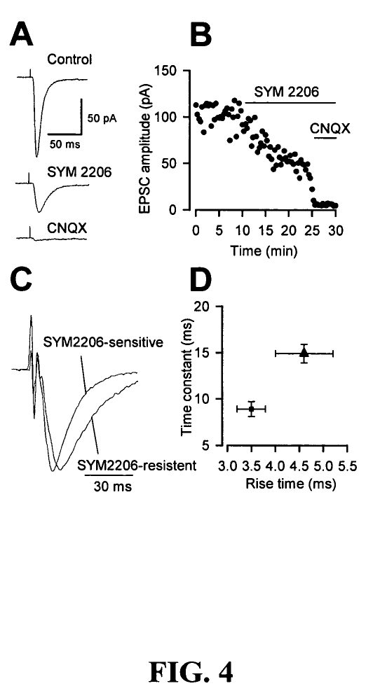

To test for possible postsynaptic KAR mediated EPSCs, whole-cell patch-clamp recordings were performed from neurons in the LA. Electrical stimulation delivered to the thalamic input (see FIG. 2A) induced fast, monosynaptic EPSCs. NMDA receptors were blocked with the selective NMDA receptor inhibitor AP5 (50 μM). The non-competitive AMPA receptor antagonist SYM2206 (Li et al., 1999) was used to separate potential KAR mediated EPSCs. SYM2206 was used at 100 μM, a concentration that produces maximal inhibition of AMPA receptors (half-maximal inhibitory concentration=1-2 μM) but less than 20-30% inhibition of KARs (Paternain et al., 1995; Wilding and Huettner, 1995). As shown in FIG. 4A, bath application of AP5 plus SYM2206 (n=17) reduced, but did not completely block, the EPSCs. The residual current was completely blocked by CNQX (20 μM) (n=5, FIG. 4A,B), indicating that the residual current was mediated by KARs. It has been reported that AMPA and KA receptor mediated currents have different activation and inactivation kinetics in spinal dorsal horn neurons and hippocampal neurons (Li et al., 1999; Cossart et al., 2002). The present results show that the rise time (10-90%) and decay time constant (τ) of KAR mediated EPSCs were significantly longer than those of AMPA-receptor mediated currents in LA pyramidal neurons (FIG. 4 C,D).

Synaptic Potentiation in the Auditory Cortex

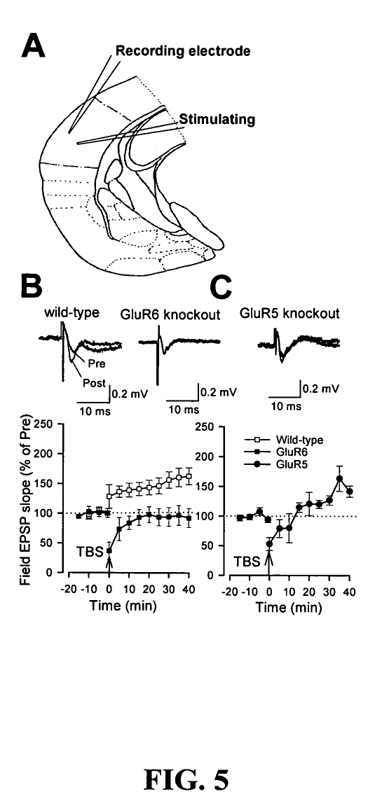

In addition to the amygdala, the auditory cortex is thought to play a role in the expression of fear memory (LeDoux, 2000). Therefore, similar recordings of LTP in slices of the auditory cortex were also performed. In slices of GluR6 knockout mice, TBS failed to induce significant potentiation (89.9±12.1%; n=5 slices/5 mice;) as compared to that of wild-type mice (147.0±10.0%; n=7 slices/7 mice; p<0.005; FIG. 5B). In slices of GluR5 knockout mice, however, TBS induced synaptic potentiation (142.0±11.2%; n=7 slices/5 mice; p<0.05 to baseline or GluR6 knockout mice; FIG. 5C).

KARs and the Amygdale

KARs contribute to synaptic transmission in the amygdala, a structure important for fear memory (Li and Rogawski, 1998). Li et al (2001) demonstrated the involvement of KARs in homosynaptic and heterosynaptic potentiation, and results using selective pharmacological agents indicated that GluR5 plays an important role in this plasticity. In the present study, LTP was induced in the lateral amygdala of adult mice using two different standard protocols for LTP: TBS (field EPSP recording) and the pairing of synaptic activity with postsynaptic depolarization (whole-cell patch-clamp recording). It was determined that the deletion of GluR5 did not affect synaptic potentiation in the amygdala. However, in GluR6 knockout mice, LTP induced by two different protocols was blocked. The activation of GluR5 KARs was shown to affect inhibitory transmission in the amygdala (see Braga et al., 2003). The LTP reported here is likely to be independent of inhibitory transmission, since inhibitory transmission was completely blocked in the whole-cell patch-clamp recordings.

In the present study, it has been determined that GluR6, but not GluR5, contributes to fear memory. Consistent with behavioral findings, LTP in the amygdala of GluR6, but not GluR5, knockout mice was significantly reduced. The defect in fear memory observed in GluR6 knockout mice is unlikely due to changes in nociception, since responses to acute and inflammatory pain were comparable to that of wild-type mice.

1. A method of treating a condition associated with fear memory in a mammal, wherein the method comprises the step of selectively inhibiting GluR6 receptors in the mammal.

2. A method as defined in claim 1, comprising administration of a selective GluR6 inhibitor to the mammal.

3. A method as defined in claim 2, wherein the inhibitor is administered in an amount ranging from 1-10 mg/kg per day.

4. A method as defined in claim 2, wherein the inhibitor is administered in combination with at least one pharmaceutically acceptable carrier.

5. A method as defined in claim 2, wherein the inhibitor is administered by injection.

6. A method of screening for a drug candidate useful to treat in a mammal a condition associated with fear memory, comprising:
a) assaying a compound for interaction with GluR6;
b) assaying the compound for interaction with at least one other kainate receptor; and
c) comparing the interaction of the compound with GluR6 and the at least one other kainate receptor, wherein interaction with GluR6 but not with said other kainate receptor indicates that the compound is a GluR6 selective drug candidate.

7. A method as defined in claim 6, wherein the interaction is a binding interaction.

8. A method as defined in claim 6, wherein the interaction is an inhibitory interaction.

9. A method as defined in claim 6, wherein the interaction is electrophysiological and modulates ion channel activity.

10. A method as defined in claim 6, wherein the GluR6 is in an animal model.

11. A method of screening for a drug candidate useful to treat in a mammal a condition associated with fear memory, comprising:
1) incubating a first mixture of labelled ligand with a GluR6 receptor-producing cell or with a membrane preparation derived therefrom followed by incubation with a drug candidate;
2) incubating a second mixture of labelled ligand with another kainate receptor-producing cell or with a membrane preparation derived therefrom followed by incubation with a drug candidate; and
3) measuring the amount of labelled ligand in the first and second mixtures that is displaced following incubation with the drug candidate, wherein a concentration of displaced labelled ligand in the first mixture as compared with the second mixture is indicative of selective GluR6 inhibition.

12. A method as defined in claim 1, wherein the condition associated with fear memory is selected from the group of depression, anxiety and psychoses.

13. A method as defined in claim 6, wherein the condition associated with fear memory is selected from the group of depression, anxiety and psychoses.

14. A method as defined in claim 11, wherein the condition associated with fear memory is selected from the group of depression, anxiety and psychoses.
